(19) United States
(12) Patent Application Publication
(10) Pub. No.: US 2007/0061006 A1
(43) Pub. Date: Mar. 15, 2007

(54) Methods of making shape memory films by chemical vapor deposition and shape memory devices made thereby

(76) Inventors: Nathan Desatnik, San Antonio, TX (US); David G. Rosenbaum, Glencoe, IL (US)

Correspondence Address: David G. Rosenbaum;ROSENBAUM & ASSOCIATES, P.C., Suite #380, 650 Dundee Road, Northbrook, IL, 60062, US

(21) Appl. No.: 11/226,548

(22) Filed: Sep. 14, 2005

(51) Int. Cl. A61F 2/06; A61L 33/00; C23C 16/00; B05D 3/00

(52) U.S. Cl. 623/1.42; 427/2.25; 427/2.21; 427/248.1

(57) ABSTRACT

A method of depositing shape memory or superelastic thin films by chemical vapor deposition (CVD) and medical devices made thereby, including stents, grafts, stent-grafts, stent covers, occlusive and filter membranes and drug-delivery devices. The method entails a thin film is deposited on a substrate surface using a CVD reaction in the production of a film of nickel-titanium shape memory or superelastic alloy. Such nickel-titanium-based shape memory or superelastic alloys may be binary nickel-titanium alloys or may include additional compounds to form ternary, quaternary, or higher level alloys.

### BACKGROUND OF THE INVENTION

The present invention generally relates to a method of depositing shape memory or superelastic thin films by chemical vapor deposition (CVD) and devices made thereby. In particular, the invention relates to a method of depositing thin films whereby a thin film is deposited on a substrate surface using a CVD reaction in the production of a film of nickel-titanium shape memory or superelastic alloy. Such nickel-titanium-based shape memory or superelastic alloys may be binary nickel-titanium alloys or may include additional compounds to form ternary, quaternary, or higher level alloys.

The present invention further relates to shape memory devices fabricated by CVD, and, in particular, implantable medical devices including stents, stent-grafts, stent covers, grafts, occlusive and filter membranes and drug-delivery devices.

CVD processes are generally associated with fabrication of microelectronic devices and components, such as integrated circuits. In recent years, physical vapor deposition (PVD) processes have been employed to fabricate shape memory alloys suitable for use in medical device applications. For example, sputtering methods have been used to produce high-strength nickel-titanium SMA films which are well suited for use as an implantable medical device. See, e.g., U.S. Patent Application Publication No. 20030059640, published Mar. 27, 2003, which is commonly assigned with the present application and is hereby incorporated by reference.

While it has been found that PVD techniques have produced acceptable films for the fabrication of implantable medical devices, because it is very difficult to obtain satisfactory step coverage of very fine features, such as those with in the range of 0.25 to 1.0 μm, where such features are desired, PVD techniques may not be well suited. Generally, however, fabrication of coherent films and coherent patterned films of nickel-titanium SMA by CVD processing is not known.

Binary nickel-titanium is currently a material of choice for many medical devices, but there are challenges in fabricating the alloy to the required shape and surface finish. However, due to limitations in currently available methods of making binary nickel-titanium, improvements in the properties of the binary material, particularly in the areas of radiopacity, superelastic performance and fatigue strength, are needed.

As the range of medical devices employing nitinol expands, the demands also increase for better material properties, ingot-melting technology, improved forming methods, superior finish and fabrication techniques. The four decades of working with Nitinol industrial and medical applications has naturally seen great improvements in all of these aspects of manufacturing. However, as implantable devices proliferate with shrinking physical dimensions, so too do demands for further improvements in radiopacity, strength, fatigue and biocompatibility of this alloy family.

Conventional bulk NiTi alloys employed as the starting or precursor material for stock NiTi rods, sheets, wires or the like is made by melting NiTi alloys and the casting ingots for primary metal working. Ingot casting, however, has several challenges which lead to downstream material properties in the finished devices, including: 1. Sensitivity to Oxygen and Carbon contamination; 2. Requirements for very tight compositional control; 3. Solidification conditions to minimize micro and macro segregation, and 4. Avoidance of non-metallic inclusions.

Many of the melting procedures that have been successfully used for titanium alloy production are valid for NiTi. Currently, the most common procedure for NiTi shape memory alloys is to use vacuum induction melting (VIM) for the primary alloy preparation followed by vacuum arc melting (VAR) to improve homogeneity in microstructure. The segregation characteristics are a function of the nature of the phase diagram and the solidification rate; faster cooling rates favor smaller dendrite arm spacing which equates to minimum segregation. Fast cooling rates are also helpful in promoting a good dispersion of particulates such as carbides and intermetallic compounds. Since the VIM process uses graphite crucibles, there is possible pick-up of carbon, however, by avoiding direct contact between titanium and the graphite crucible and by holding the melting temperature below 1450° C., the carbon level can be held to 200 to 500 ppm. The general requirements on Nitinol chemistry and trace elements are defined in ASTM standard, F2063-00. The transformation temperature in a relatively small VIM ingot can generally be held to +/−5° C. Controlling micro and macro segregation becomes more difficult with increasing ingot size. To refine the microstructure, the vacuum arc melting process, VAR, is used where a consumable electrode of the VIM melted alloy is melted in a copper mold resulting in a much more homogeneous ingot with far less segregation. Noting that for alloys with greater than 55.0 at % nickel, a one percent deviation in nickel or titanium content will result in a transformation temperature change of about 100° C., analytical techniques do not have the accuracy to predict the transformation temperature. In fact, transformation temperatures and chemistry are more effectively controlled by measuring the VIM metal charge. Although it is possible to make in-situ alloy corrections during the VIM melting by analyzing samples taken from the melt, this is a difficult procedure in manufacturing.

Alternatively, multiple VAR melting practice is used for commercial NiTi ingot production. Avoiding contact with the graphite crucible, the VAR ingot tends to produce a cleaner alloy with carbon contamination typically less than 200 ppm. Unfortunately the fact that only a small molten zone is produced as the arc progressively melts the electrode, there is a less homogeneous distribution in chemistry along the ingot and the top to bottom ingot transformation may vary greater than 10° C. By repeating the VAR process, so called multiple melting, a more homogeneous ingot may result.

Although the present preferred melting system for ingot production is the VIM-VAR described, the vacuum induction skull melting process has the potential for producing an ingot of higher purity. In this process the crucible has a very unique geometry, crown like in appearance it has a water cooled base with side stakes consisting of rectangular water cooled rods, spaced at about one-half of the rod diameter. The crucible is surrounded by an induction coil, and the entire assembly is capable of being tilted to pour into molds placed within the vacuum chamber. When the alloy is melted a thin layer of solidified metal is created over the crucible bottom and the stake sides, resulting in the melt being confined in a crucible or skull of the alloy being melted, eliminating the potential for crucible-melt interaction, a minimum of contamination and very vigorous melt stirring. The shell formed over the side stakes shrinks away from direct contact, thus preventing electrical short-circuiting of the electromagnetic field. Since the electromagnetic induction is absorbed by the metal crucible as well as the melt, the process is inefficient and requires a large power source. While the benefits of high purity nitinol have not yet been proven, there is evidence to suggest that impurities in the nitinol interfere with the biological response to implanted nitinol devices and can be speculated that they will have an impact on their thermodynamic and mechanical properties.

Ternary alloys, such as NiTiCu and NiTiNb, have been found to experience smaller changes to their transformation temperatures with respect to compositional variation. However, Cu addition leads to hot shortness, a problem in hot conversion while Nb enlarges transformation hysteresis. Other alloying possibilities that reduce compositional sensitivity and improve superelastic properties may exist which would make the ingot process more efficient. Moreover, copper is contraindicated in implantable medical devices as it is cytotoxic in vivo.

With the exception of investment cast implantable devices such as orthopedic systems, most medical devices are fabricated from wire, strip or tube. As such, the primary processing of the ingot involves a hot break down by either hot forging or rolling. The hot working breaks down the cast structure and provides an acceptable size for further cold drawing. Hot working must be carried out at a temperature which avoids severe oxidation. For nitinol the preferred hot working temperature is 800 degrees C., a temperature which ensures good workability and minimizes oxide buildup. Hot rolling employing a mill with grooved rolls is the most commonly used break down process.

Once a rod of suitable dimension has been achieved, nitinol is cold worked to yield a final dimension and, by combining the cold working with heat treatment, achieve the desired mechanical and physical properties. Nitinol has a very high work hardening coefficient, which limits the cold reduction achievable in a single pass. Interpass annealing is carried out at 600 to 800 degrees C., and in most cases the oxide formed is not removed until the final pass since its presence assists in retaining the die lubricant. Lubricants which are commonly used include graphite containing water, molybdenum disulfide, oil based lubricants and sodium stearate soap. Lubricant must be scrupulously removed after the final dimension has been achieved. Round wire is produced in single or multiple die stands, and rectangular wire is produced using a Turk's head bull block process. Round wires can be flattened to yield ribbon, although tolerances are better in the Turk's head drawing process. Nitinol tube has become the major starting point for the production of stents, and, as such, there are several variations on tube drawing processes: floating mandrel, non-deformable mandrel and deformable mandrel. The details of tube drawing methods are proprietary, however several are described in patents. Tubes with an O.D. as small as 0.25 mm are being produced. The problems which are being addressed include tube concentricity, tube outer surface finish and tube I.D. cleaning. As in wire drawing, interpass annealing is required, and surface oxidation is minimized by annealing in an inert atmosphere. Cleaning is particularly important when the tube is to be laser cut since impurities can be incorporated into the recast structure and promote micro cracking before the recast structure is removed.

Machining of nitinol is very difficult due to its very rapid hardening. Although with proper carbide tooling and control of tool geometry, speed and feed, excellent tolerance and finish can be achieved in turning operations. Milling with its interrupted cut is more difficult with tool breakage being a frequent problem. Drilling, as with turning, requires careful control of feed and speed, and the use of chlorinated lubricant is recommended. Taping is extremely difficult and is not recommended. Cylindrical centerless grinding is a useful process for developing a good surface on tubing and wire, and is used for creating a tapered end on catheter guide wires. Other abrasive methods such as abrasive wheel cut off and abrasive water jet cutting are also used in processing nitinol. Electrodischarge machining (EDM) is quite useful, although not really suitable for volume production. Since a recast layer is developed by the high energy spark, this contaminated layer containing electrode copper and oxides is usually removed. As we mentioned earlier, photoetching is used in special cases for forming stents, filters and baskets. Once the tooling has been prepared, consisting of a photographic image, or multiple images, the process is capable of efficient parts fabrication. Tolerance in the process is 10% of the metal thickness, thus for a 0.025 mm sheet, the tolerance would be +/−0.0025 mm, quite adequate for most components. Three-dimensional structures such as stents can be fabricated by the photoetching process using novel imaging techniques. For example, using contact film and an elliptical mirror or an optical scanning system in synchronization with numerical controlled part rotation and motion, a desired pattern can be imprinted on the photo-resistance coating on a cylindrical structure before etching.

Laser cutting and machining has become a preferred method for creating stents from nitinol tube. Very complicated geometries are produced using computer controlled part motion and finely focused pulsed Nd:YAG laser beams. Since laser-cutting is essentially a melting process, a recast layer is produced on the cut surface. To prevent surface contamination, the part must be very clean before laser cutting is initiated. The recast layer is susceptible to micro-cracking and must be removed to ensure a good fatigue life for the machined component. A heat affected zone (HAZ) is also present and must be removed in post cutting operations. The usual techniques for removing the recast materials and HAZ include electro-polishing, abrasive and vapor blast cleaning. Laser cutting is fast and very flexible, and cut geometry is readily changed through reprogramming of the computer control.

Nitinol materials in either the cold worked or heat-treated state can be easily sheared or stamped, but they are difficult to form to an accurate geometry, whether by forming wire shapes or die pressing. The major problem, spring-back, is significant at ambient temperature. To counteract spring back the part can be over deformed so that on release of stress the desired shape is achieved. Unfortunately this leads to the formation of stress-induced martensite, which will degrade the desired mechanical properties and shift the transformation temperature. The solution to spring back is to heat treat the part under constraint. Where high volume fabrication is required the use of many constraining jigs may be necessary. In another approach to volume production the part can be formed on an insulated mandrel and heated electrically for a few seconds, and then dropped from the mandrel. Only simple shapes can be processed in this manner.

For the broad range of medical devices which use superelastic Nitinol, the initial condition before heat treatment is 30 to 40% cold work, followed by heat treatment at 500 degrees C. For shape memory alloys, the heat treatment range is preferably 350 to 450 degrees C. Good superelastic performance can also be generated by a solution treatment and aging of alloys with greater than 55.5% nickel. For this treatment, the solution treating temperature is between 600 to 900 degrees C. and the aging treatment is carried out at 400 degrees C. This procedure is useful where forming of a complicated nature can be carried out with the ductile solution treated alloy and then rendered superelasticity by aging. The aging process causes precipitation of the Ni-rich intermetallic compound, and since this depletes the matrix of Ni, there is a concomitant increase in the transformation temperature of the resulting material.

In the early development of nitinol actuators and components, joining was confined to mechanical fastening by crimping, riveting and swaging. Recent development in laser welding processes has made joining of nitinol to itself a routine process. Contamination by oxygen and nitrogen make it essential to carry out any joining process involving melting under either a vacuum or an inert gas cover. CO₂ and ND:YAG lasers are both capable of producing welds with excellent strength retention. The CO₂ laser welds do exhibit reduced strength and resistance to permanent deformation in the fusion zone and the HAZ. This is also true of welds made using the tungsten inert gas (TIG) process. Electron beam welding is also useful for welding smaller parts, although the process is slow by reason of the need to load and unload through a vacuum port. The best welds seem to be made using the Nd:YAG laser with a yield of 75% of the base metal strength, and only 0.2% permanent deformation after a 7% strain of a superelastic weld specimen. Resistance welding can also be used, again with adequate inert gas shielding. The Ti rich alloys are more susceptible to weld cracking, although using a consumable wire crack free welds with good strength can be produced.

The adherent oxide surface formed on Nitinol is a barrier to conventional soldering processes. A halogen based flux, as described in U.S. Pat. No. 5,242,759, makes possible soft soldering. An intermediate barrier of nickel, produced by electroplating or electroless nickel deposition creates a surface which will accept solder with mild flux. Another approach is to electroless plate a copper layer using a copper fluoroborate-HF solution, and follow this with a nickel plating. In some cases a second metal layer, such as gold, over the nickel improves the solderability. Ultrasonic soldering, developed for removing the oxide from aluminum to promote solder wetting, has also been used successfully on Nitinol with a Sn based solder.

There are cases where it is desirable to join Nitinol to other metals, for example, stainless steel. Brittle intermetallic compounds are formed in the fusion zone of such welds, rendering them useless. To avoid the formation of the intermetallics such as FeTi and Fe₂Ti some intermediate layer must be employed which is compatible with both Nitinol and stainless steel. Tantalum is one metal which has the desired characteristics and welds have been successful with this metal. Other intermediate layers which can produce similar results are Nb and Mo. Both resistance welding and percussive arc butt welding with a Ta interlayer have yielded acceptable joints.

During hot working of nitinol an oxide is formed and may be removed prior to cold working using grit blasting, polishing or chemical means. Hydrogen fluoride cleaning is highly effective in cleaning the oxide layer, but care must be taken when cleaning fine wires or thin wall tubes to avoid significant changes in the desired dimensions of the workpiece. Mechanical polishing, such as vibratory finishing can produce a mirror finish in batch processing. Trial polishing is necessary in order to select the correct medium for the particular part. Electropolishing is a very effective technique for producing a very smooth finish, although consistent results depend heavily on electrolyte and polishing parameters such as voltage and temperature. Mixtures of perchloric acid with acetic acid or sulfuric acid in methanol have been also used with good results,

Corrosion resistance and biocompatibility are both affected by the final method finishing the nitinol component. Nickel leaching in vivo is dependent on the finishing technique. It has been found that a final surface of titanium oxide acts as a barrier to nickel leaching and also enhances surface passivity, and, therefore, corrosion resistance. Although the smooth appearance of a mechanically polished surface is attractive, in fact this type of surface has the poorest corrosion resistance while chemical etching enhances passivity. Electropolishing itself does not necessarily enhance corrosion resistance, however if this is followed with a passivation procedure, an optimum corrosion resistance and biocompatibility can be achieved.

Metallic and organic coatings can be applied to nitinol by a variety of methods, however, such coatings are generally not desirable due to the difficulty in obtaining good adhesion and sufficient ductility to avoid flaking when the workpiece is in service. Moreover, a damaged metallic coating can also lead to galvanic corrosion.

Powder metallurgy (PM) is an important route to making precision, high strength ferrous and non-ferrous parts in a very efficient manner. Methods of atomizing have been developed for producing powders of non-reactive and reactive metals, with excellent control of chemistry. When applied to Nitinol the major problem is control of the oxygen content. Typical PM Nitinol parts may have as much as 3000 ppm of oxygen. Although by careful handling of the powders oxygen levels can be reduced to the region of 1500 ppm, there is still concern for the effect on fatigue and ductility.

Alternate methods of fabricating shape memory alloy nickel-titanium include atomization of pre-alloyed Nitinol, which has been carried out using gas atomization, hydriding, pulverizing and mechanical alloying. Blended powders are compacted by hot and cold isostatic pressing, hot and cold uniaxial die compaction and direct powder rolling. A unique process was developed in which powder is loaded into a pyrex glass tube, vibrated for compaction, and then evacuated and sealed. The tube is placed in a vertical furnace where sintering takes place with atmospheric pressure supplying the positive pressure. The product has 68% porosity and can be further processed by hot isostatic pressing to achieve densities of greater than 95%. Hot working by rolling or swaging can yield fully dense material. By blending in the proper ratio of two nitinol powders with different transformation temperatures one can obtain a sintered product with accurate control of a targeted transformation temperature. The process does not, however, prove to be competitive with VIM/VAR processing.

Micro electromechanical systems (MEMS) have received a great deal of attention for control of fluidic devices and for applications in robotic systems. Interest in the application of thin film Nitinol MEMS to medical device fabrication has also seen rapid growth. Films can be deposited on metallic, silicon, glass and polymer substrates. Where deposited on cool conductive substrates the deposit is amorphous and to develop shape memory properties the film must be heated to cause crystallization. Although a variety of substrates are used, silicon has appeared in the literature as the preferred material. A variety of Ni—Ti alloys have been studied as thin film deposits; the two most frequently employed for MEMS devices are binary NiTi and NiTiCu. Pre-alloyed targets as well as elemental targets are used, although the former produces more uniform compositions. Several types of miniature valves have been developed using the thin film to open and close an orifice in the silicon substrate. A miniature pump system capable of high frequency and high pressure has been produced using a unique deposition technique to produce a thin film with a composition gradient which varies from equiatomic NiTi to a Ni rich composition. The Ni-rich layer of the film acts as a bias to provide two-way memory actuation. When heated, the film rises to form a dome and when cooled the film returns to a flat position; thus a heating/cooling cycle can generate a pumping action. MEMS pumps and valves can form the basis for a variety of drug delivery systems and devices of this type that will certainly achieve use in the future.

Physical vapor deposition techniques, such as evaporation and sputter deposition processes, have been used to fabricate tubular films useful as stent covers and/or grafts and have the potential to produce full-sized stents. The parameters that characterize these deposition processes can be manipulated to produce film with controlled composition, grain structure, and topography as well as desired thermodynamic and mechanical properties. These deposition techniques have been employed to produce biocompatible materials.

The deployment of stents is aided by observing the position of the stent by radiograph, however, binary Nitinol has relatively poor radiopacity and its image is difficult to see. Stents with coatings of higher atomic number materials such as gold improve radiopacity and provide a clearer image. An alternative has been disclosed in which Nitinol with ternary additions of elements such as platinum, palladium or other elements with high atomic number is rendered more radiopaque. These ternary alloys still retain the desired superelastic characteristics of the binary nitinol.

Chemical vapor deposition generally refers to the formation of a non-volatile solid film on a substrate from the reaction of vapor phase chemical reactants containing the right constituents. A reaction chamber is used for this process, into which the reactant gases are introduced to decompose and react with the substrate to form the film. A basic CVD process consists of the following steps: 1) a predefined mix of reactant gases and diluent inert gases are introduced at a specified flow rate into the reaction chamber; 2) the gas species move to the substrate; 3) the reactants get adsorbed on the surface of the substrate; 4) the reactants undergo chemical reactions with the substrate to form the film; and 5) the gaseous by-products of the reactions are desorbed and evacuated from the reaction chamber. During the process of chemical vapor deposition, the reactant gases not only react with the substrate material at the surface, but also in the gas phase in the reactor's chamber. Reactions that take place at the substrate surface are known as heterogeneous reactions, and are selectively occurring on the heated surface of the substrate where they create good-quality films. Reactions that take place in the gas phase are known as homogeneous reactions. Homogeneous reactions form gas phase aggregates of the depositing material, which adhere to the surface poorly and at the same time form low-density films with added defects. Hence, heterogeneous reactions are more desirable than homogeneous reactions during chemical vapor deposition.

Chemical vapor deposition of thin films involves gas-phase and surface reactions combined with transport processes. Precursor gases, which are often diluted in inert carrier gases, are delivered to a reaction chamber at an appropriate temperature to maintain a high quality vapor. As they pass over or come into contact with a heated substrate, they react or decompose to form a solid phase which is deposited onto the substrate. Substrate temperature is typically critical as it influences the rate of film formation, as well as the properties of the deposited material. Chamber temperature control is significant because it can be used to control formation of gaseous reactants or depletion of reactants to the chamber walls. Formation of gaseous reactants may also be controlled by controlling the chamber pressure. The process of chemical vapor deposition may be summarized in the following elements of the film growth process:
1. mass transport in the bulk gas flow region from the reactor inlet to the deposition zone;
2. mass transport of film precursors to the growth surface;
3. adsorption of the film precursors on the growth surface;
4. surface reactions of the adsorbed species on the surface of the substrate;
5. incorporation of film constituents into the growing film island;
6. desorption of byproducts of the surface reactions; and
7. mass transport of byproducts in the bulk gas flow region away from the deposition zone towards the reactor outlet.
   An excellent monograph describing fundamentals of CVD processing and different CVD reactor types is Hess, D. W. and Jensen, K. F., eds, Microelectronics Processing: Chemical Engineering Aspects, Advances in Chemistry Series Vol. 221, 1898 pp. 199-263 (1989) published by the American Chemical Society as ISBN 0-8412-1475-1, which is hereby incorporated by reference.

A typical CVD system consists of the following basic components:
a. a gas delivery system which supplies and controls the flow of gases to a reactor chamber;
b. a reactor chamber in which the deposition occurs;
c. a substrate loading mechanism which is a system for introducing and removing substrates into the reactor chamber;
d. a source which provides energy or heat sufficient to react or decompose the precursor gases, such as, for example, plasma glow discharge for PECVD, or alternative energy sources such as heating sources, radiant heating sources, induction or RF heating sources, UV-visible light, or lasers;
e. a vacuum system which maintains low chamber pressure, provides for a short residence time of gaseous species in the chamber and evacuates byproducts of the deposition; and
f. process control equipment which monitors and controls process parameters of the deposition, such as temperature, pressure, duration and flows.

While it is known that nickel films and titanium films may be produced by CVD processing, heretofore, it has been unknown that nickel-titanium SMA films may also be produced by CVD processing. Nickel films have been produced by first making gaseous nickel carbonyl (Ni(CO₄) from the reaction product of nickel powder and carbon monoxide (CO). Nickel carbonyl is a highly volatile, carcinogenic gas that has been used as an intermediate species in purification of nickel from nickel-containing ores by the Mond reaction. However, because the bonds between the nickel and the carbon monoxide groups are week, the nickel carbonyl rapidly disassociates back to the nickel metal and carbon dioxide under a slightly elevated temperature, thus making it a fine nickel donor candidate for CVD processing.

Titanium films have been deposited by CVD processing by forming intermediate titanium halides, such as titanium tetrachloride (TiCl₄) (See, e.g., U.S. Publication No. 2001/0021414 published Sep. 13, 2001, hereby incorporated by reference) or titanium tetraiodide (TiL₄) as the source gas to deposit salt-free titanium, titanium nitride (TiN), titanium silicon nitride (TiSiN), titanium disulfide (TiS₂) [See, e.g., Kikkawa, S., et al. “Titanium Disulfide Thin Film Prepared by Plasma CVD”, J. Mater. Res., Vol. 5, No. 12, p. 2894 (1996)], and titanium boride (TiB₂). See, e.g., Hendricks, J. H., et al, “Metal and Ceramic Thin Film Growth by Reaction of Alkali Metals with Metal Halides: A New Route for Low Temperature CVD” at www.nist.gov/sigmaxi/Posters98/abs/Hendricks.html, or by decomposition of tris-(2,2′bipyridine) titanium. See, e.g., Pierson, H. O., Handbook of Chemical Vapor Deposition (CVD): Principles, Technology and Applications, 2ed, 1999, which is hereby incorporated by reference. Hendricks, et al. describe a low temperature CVD method for the growth of metal and ceramic thin films using a low pressure coflow diffusion flame reactor to react alkali metal vapor with metal halide vapor. The reaction chemistry is described by the following general equation:
(mn)Na+nMXm→(M)n+(nm)NaX
where Na is sodium vapor, or other alkali metal, (or another alkali metal) and MXm is a metal-halide (M=metal or other element such as Si, or C, and X=halogen atom, and m and n are integers). In this reaction, the alkali metal strips halogen from the metal halide. The metal is then free to grow into a thin film on a substrate placed in the reaction zone. Metal nitride or metal oxide ceramic films are easily formed by the introduction of nitrogen or oxygen gases into the reactor. Using the precursors of sodium metal vapor, titanium tetrachloride (the limiting reagent), and either Ar or N₂ gases, salt-free titanium (Ti), titanium nitride (TiN), and titanium silicide (Ti₃Si₅, TiSi₂) thin films have been grown on copper and silicon substrates. This technique produced salt-free titanium and titanium nitride thin films on copper substrates heated to 600° C., a temperature significantly lower than the 900° C. to 1200° C. required for conventional thermal CVD of titanium. A composite salt/Ti film was grown on a silicon wafer at 260° C., while at 600° C., a salt-free titanium silicide thin film was produced.

Nickel films have been chemical vapor deposited by the thermal decomposition reaction of nickel carbonyl, Ni(CO)₄, by hydrogen reduction of nickel alkyl, Ni(C₅H₅)₂ (NiCp₂, bis(cyclopentadienyl)nickel, nickelocene)/H₂ at about 200 degrees C., by reduction of Nickel II 2,4 pentanedionate (Ni (C₅H₇O₂)₂, by hydrogen reduction of nickel chelate, Ni (C₅HF₆O₂)₂. Nickel films deposited at around 200° C. showed carbon content lower than 5% and lower resistivity because of the effective dissociation of Ni-Cp and desorption of Cp from the surface. Nickel films deposited with hydrogen addition showed higher purity, crystallinity, and lower resistivity due to the removal of the carbon on the surface. See, e.g., Kang, J. K., et al., Metalorganic chemical vapor deposition of nickel films from Ni(C₅H₅)₂/H₂″ J. Mater. Res. Aug. 2000, found at: www.mrs.org/publications/jmr/jmra/2000/aug/029.html.

TiN films have been deposited using plasma-enhanced CVD, in which a plasma is generated with a gaseous mixture of nitrogen (N₂) and hydrogen (H₂), a reaction gas mixture of TiCl₄, N₂, and H₂ is then introduced into the reactor, the TiCl₄ or precursor is nitrided by the active nitrogen ions and atoms generated by the plasma, and a TiN film is deposited on the substrate. Titanium tetrachloride (TiCl₄) is used as the reactive gas in the methods described above because the surface of the deposited film is smooth, and the step coverage is excellent.

In extensive work with physical vapor deposition of nickel-titanium binary and ternary shape memory alloys, it has been learned that the deposition process may be controlled in such a manner as to deposit a film having shape memory or superelastic properties within tightly controlled transition temperatures without the need for post-deposition precipitation annealing. In metals containing another element in a supersaturated solid solution, such as nickel-titanium SMA, under annealing conditions, the solution precipitates a compound with the solvent metal. In the presence of a dislocation, atoms precipitate into the dislocation at a rate of:

$$n ( t ) ∝ {( \frac{t}{T} )}^{2 / 3}$$

where n(t)=the number of atoms precipitating over time, t., and T=temperature. Precipitation annealing is useful for locking dislocations and preventing them from moving through the material lattice which hardens the material, and is, therefore, useful in construction materials. However, precipitate formation creates internal stresses that may significantly weaken crystal lattices, which is problematic for shape memory behavior of MEMS devices using metallic compounds. See, e.g., Stark, B., ed., MEMS Reliability Assurance Guidelines for Space Applications, JPL Publication No. 99-1, National Aeronautics and Space Administration, Jet Propulsion Laboratory, California Institute of Technology, p. 31, January, 1999.

CVD reactions are generally of several different types: pyrolysis, reduction, oxidation or compound formation.

Pyrolitic or thermal decomposition reactions are exemplified by the following reaction:
AB(g)→A(s)+B(g)
An example of a typical CVD pyrolitic reaction is silicon deposition from silane at about 650 degrees C.:
SiH₄(b)→Si(s)+2H₂
Pyrolysis reactions are typically used to deposit Al, Ti, Pb, Mo, Fe, Ni, B, Zr, C. Si, Ge, SiO₂, Al₂O₃, MnO₂, BN, Si₃N₄, GaN, Si₁₋ₓ Geₓ, etc.

Reduction reactions are typically conducted at temperatures lower than pyrolysis reactions and often use hydrogen gas. The reduction reactions are reversible and may be used for substrate or reactor cleaning. A typical reduction reaction is exemplified by the following reaction:
WF₆(g)+H₂(g)←→W(s)+6HF(g)
Reduction reactions are typically used to deposit Al, Ti, Sn, Ta, Nb, Cr, Mo, Fe, B. Si, Ge, TaB, TiB₂, SiO₂, BP, Nb₃Ge, Si₁₋ₓ Geₓ, etc.

Oxidation reactions typically are carried out in the presence of oxygen gas and are exemplified by the following reaction:
AX(g)+O2(g)→AO(s)+[O]X(g)
An example of a typical oxidation reaction is silicon dioxide decomposition from silane and oxygen at about 450 degrees C.:
SiH₄(g)+O₂(g)→SiO₂(s)+2H₂(g)
Oxidation reactions are typically used to deposit SiO₂, Al₂O₃, TiO₂, Ta₂O₅, SnO₂, ZnO, etc.

Finally, compound formation typically employs either ammonia or water vapor and is exemplified by the following reactions:
AX(g)+NH₃→AN(s)+HX(g)
AX(g)+H₂O→AO(s)+HX(g)
Boron nitride films have been deposited as wear resistant films at 1100 degrees C. using compound formation reactions as follows:
BF₃(g)+NH₃→BN(s)+3HF(g)
Compound formation reactions may be used to deposit TiN, TaN, SiC, Al₂O₃, In₂O₃, SnO₂, SiO₂, or similar materials.

CVD processes include, for example, silicon epitaxy, low-pressure CVD (LPCVD), vapor-phase epitaxy (VPE), metalorganic CVD (MOCVD), plasma-enhanced CVD (PECVD), photon-assisted CVD (PACVD) also known as laser-assisted CVD (LACVD). In producing the inventive SMA nickel-titanium films, PECVD, thermal CVD, and LACVD are considered the most suitable. Examples of thermal CVD are MOCVD and LPCVD.

LPCVD is typically carried out higher temperatures as compared to PECVD which, depending upon the product requirements may be a process selection criterion. Additionally, LPCVD is typically carried out at pressures between about 1 mTorr to 1 Torr and requires higher initial gas concentrations of the reactant species. The advantages of LPCVD are that it provides better film uniformity, better film step coverage and fewer defects than PECVD processing. PECVD employs a plasma which energizes the gas molecules of the reactant species to permit higher reactivity at lower temperatures and pressures than thermal CVD or LPCVD. PECVD utilizes higher pressures than sputter deposition which reduces the energy of each ion when it reaches the cathode/substrate and results in minimal sputtering effects. RF plasma discharge may also be employed. In PECVD, control over process parameters, including substrate temperature, gas flow, pressure, power and frequency may be exercised to control the deposition and film growth. In this case, LACVD and PACVD employ laser energy to enhance surface reactivity between the gaseous species and the substrate surface. LACVD and PACVD operate by two processes. A pyrolytic process heats the substrate to enhance the reactions and a photolytic process, typically using UV irradiation, enhances gas phase dissociation to enhance reactivity. MOCVD employs organometallic source materials that achieve high vapor pressures at relatively low temperatures.

Chemical vapor deposition of a reactant gaseous species will only occur under conditions that are thermodynamically favorable. These conditions exist where the free-energy change of the chemical reaction, known as ΔGᵣ, is a negative value. To calculate ΔG_{r,} it is necessary to know the thermodynamic properties of each component, i.e., their free energies of formation, i.e., their Gibbs free energy, known as ΔG_{ƒ}. The free energy change is expressed by the following equation:
ΔGᵣ_{^{o}}=ΣΔG_{ƒ}_{^{o}} products−ΣΔG_{η}_{^{o}} reactants

The free energy of formation is not a fixed value, but is variable as a function of several parameters including the type of reactants, their molar ratio, process temperature and process pressure.

The microstructure of the deposited film may be controlled by manipulating the deposition process parameters such as temperature, pressure, supersaturation and selection of the CVD reaction. It is generally understood that pressure controls the thickness of the boundary layer and, therefore the degree of diffusion. Lower pressure deposition tends to minimize diffusion and, therefore surface kinetics become rate controlling. Under these conditions, the deposited films tend to be fine grained. Fine-grained structures may also be obtained at low deposition temperature, high supersaturation and lower pressure.

Higher deposition temperatures tend to result in columnar grain structures as a result of uninterrupted grain growth toward the reactant source. Columnar grain structures become more pronounces as the film becomes thicker. It is generally recognized that columnar grain structures are undesirable due to concomitant structural, chemical and electrical anisotropy and rapid diffusion of impurities along the grain boundaries.

While the use of physical vapor deposition processes, such as sputtering, has become known as a viable means to fabricate shape memory alloys, including nickel-titanium binary and ternary alloys, use of chemical vapor deposition processes to fabricate shape memory alloys has been, heretofore, unknown.

### SUMMARY OF THE INVENTION

In one aspect, the present invention relates to a method of depositing a nickel-titanium shape memory alloy film on a substrate by chemical vapor deposition.

It is a principle objective of the present invention to provide high strength deposited shape memory or superelastic nitinol materials. In particular, it is an object of the present invention to provide high strength vacuum deposited shape memory or superelastic nitinol films that are useful in medical, mechanical and electronic applications. A further objective of the present invention is to provide a method of making the high strength shape memory or superelastic nitinol materials. Additionally, in view of the difficulties in maintaining desired Aₚ values, the present invention provides a method of forming shape memory or superelastic nitinol materials having desired transition temperature values without employing precipitation annealing.

As used herein, the term “nitinol” is intended to encompass shape memory or superelastic nickel-titanium alloys. Such alloys may include other materials in functional amounts, such as tantalum, to achieve desired properties, such as, for example, improved radio-opacity.

As used herein the terms “shape memory material” or “superelastic material” are intended to include metal alloys and non-metallic materials exhibiting shape memory or superelastic mechanical properties. Examples of suitable metal alloys include alloys of titanium, vanadium, aluminum, nickel, tantalum, zirconium, chromium, silver, gold, silicon, magnesium, niobium, scandium, platinum, cobalt, palladium, manganese and/or molybdenum. Shape memory polymers are composed of two components with different thermal characteristics. For example, a known shape memory polymer comprises oligo(ε-caprolactone)diol and crystallisable oligo(ρ-dioxanone)diol, each already used separately in clinical applications such as drug delivery. The biodegradable multiblockcopolymer features two blockbuilding segments, a hard segment and a “switching” segment, which are linked together in linear chains. The higher-temperature shape is the plastic's “programmed” or “permanent” form, which it assumes after heating.

In accordance with another aspect of the invention, the inventive method includes a plasma-enhanced chemical vapor deposition process including the steps of introducing an inert gas into a plasma-enhanced CVD reactor; generating a plasma in the reactor; introducing a reaction gas comprising a titanium compound and a nickel compound susceptible of chemical vapor deposition onto a substrate, and depositing a NiTi film on a substrate in the reactor. The plasma is preferably generated with an inert gas prior to introduction of the reactants.

In accordance with an alternate embodiment of the present invention, the inventive method includes a laser-enhanced chemical vapor deposition process including the steps of introducing a reaction gas comprising a titanium compound and a nickel compound susceptible of chemical vapor deposition onto a substrate into a LECVD reactor, irradiating a region of the substrate by laser energy appropriate for co-deposition of nickel and titanium onto the substrate in the local region of the applied laser energy, thereby depositing a NiTi region onto the substrate at the localized hot spot created on the substrate by the applied laser energy.

In accordance with a further embodiment of the present invention, there are provided shape memory or superelastic devices in which the device is formed by CVD deposition of nickel-titanium to form the device geometry. Post-deposition processing is then employed to modify the formed device to render it suitable for its intended use. For example, drug-eluting stents or drug-eluting membranes may be formed by CVD. Such devices will consist of a generally tubular device geometry having luminal and abluminal wall surfaces, opposing end surfaces continuously interconnecting the luminal and abluminal wall surfaces and at least one interior chamber between the luminal and abluminal wall surfaces. The generally tubular device geometry is formed by CVD deposition of shape memory or superelastic materials which forms the luminal and abluminal wall surfaces and the opposing end surfaces onto a generally tubular sacrificial substrate. Post-deposition removal of the sacrificial substrate, such as by chemical etching, through openings formed through the luminal or abluminal wall surfaces or the opposing end surfaces, forms the at least one interior chamber within the generally tubular device, which is concentrically positioned relative to a central lumen of the generally tubular device.

Alternatively, shape memory or superelastic films may be deposited by CVD onto substrates which are intended to form part of the resulting device. For example, a polymeric balloon, catheter, guidewire, pre-existing stent, pre-existing graft, or the like may be employed as the substrate with formation of a shape memory or superelastic film directly onto the pre-existing device/substrate.

These and other features and advantages of the present invention will become more apparent to those of ordinary skill in the art from the following more detailed description of the present invention with reference to preferred embodiments thereof.

### BRIEF DESCRIPTION OF THE FIGURES

FIG. 1 is a schematic diagram illustrating a prototypical chemical vapor deposition reactor of the type suitable for use in practicing the present invention.

FIG. 2 is a process flow diagram illustrating the method of the present invention.

FIG. 3 is a perspective view of a forming substrate employed in the method of the present invention.

FIG. 4 is a perspective view of an unpatterned double-walled CVD deposited film of the inventive implantable drug-eluting device fabricated in accordance with the present method.

FIG. 5 is a cross-sectional view taken along line 5-5 of FIG. 4.

FIGS. 6A-6D are sequential diagrams illustrating the sequential steps in CVD fabrication of an implantable drug-eluting device in accordance with the present invention.

FIGS. 7A-7D are transverse cross-sectional views taken along lines 7A-7A, 7B-7B, 7C-7C and 7D-7D of FIGS. 7A-7D, respectively.

### DETAILED DESCRIPTION OF THE PREFERRED EMBODIMENTS

The present invention includes an inventive high-strength shape memory and/or superelastic nitinol material as well as a process for fabricating the thin-film shape memory and/or superelastic nitinol materials, including the inventive graft material. The inventive material is characterized by having high mechanical strength and toughness exceeding that found in the art and does not require precipitation annealing to either shape set or set the transition temperature of the material. In accordance with the method of the present invention, the inventive nitinol materials may be produced for certain intended end-uses, such as MEMS and medical devices, using deposition technologies including but not limited to PVD, sputter deposition, plasma deposition, ion beam deposition or the like to form the film, and post-deposition use of etching, photolithography, machining, or ablation techniques to fashion the deposited film for an intended end-use. In addition to depositing shape memory nitinol materials in their martensite state and having Aₛ values below body temperature so that they transition to the austenite phase upon in vivo introduction, the method of the present invention may be employed to deposit nitinol materials in a martensite state having Aₛ values greater than body temperature so that these materials exhibit martensitic behavior in vivo. Alternatively, the method of the present invention may be employed to deposit nitinol materials in an austenite phase having an Aₛ value sufficiently low so as to behave completely austeniticly in vivo. A significant aspect of the method of the present invention, is that the method succeeds in depositing materials such that the stoichiometry of the nickel-titanium alloy is such that no heat setting is required to impart a desired transition temperature value, rather the materials as deposited using the invention method have predetermined transition temperature values imparted as a result of manipulation of the method parameters described.

By employing vacuum deposition methodologies, one is able to form materials directly into a desired 2D or 3D geometry, e.g., planar, tubular, or multi-surfaced geometries. The common principle of the deposition processes is to take a material in a minimally processed form, such as pellets or thick foils (the source material) and atomize them. The term atomization is used here loosely to include forming atomic or molecular size particles, both charged and/or neutral and both comprised of a single atom and/or of a cluster of atoms. Atomization may be carried out using heat, as is the case in PVD, or using the effect of collisional processes, as in the case of sputter deposition, for example. The atoms or particles of the source material then deposit on a substrate or mandrel to form the desired material. In most cases, the deposited material is then either partially or completely removed from the substrate, to form the desired product.

Without limiting the scope of application of the present invention, the following are specific examples of products or devices which may be fabricated using the present invention: implantable stents, nitinol grafts, stent-graft devices in which either or both components are fabricated from the inventive nitinol material, general purpose seamless nitinol tubes, sheets, films or foils which may be, for example, employed as MEMs devices.

In accordance with the present invention, an implantable endoluminal graft is provided that is comprised of two main features: a microporous metal thin film covering and an underlying structural support member, which are physically connected to one another. The implantable endoluminal graft has a delivery profile that allows for uncomplicated entry and passage throughout an anatomical passageway, more particularly a vascular system. Additionally, the implantable endoluminal graft is formed from a shape memory material, preferably nitinol, which permits the graft to expand in vivo to support a lumen wall.

The term “pseudometal” and “pseudometallic material,” as used herein, is defined as a biocompatible material which exhibits biological response and material characteristics substantially the same as biocompatible metals. Examples of pseudometallic materials include, for example, composite materials, ceramics, quartz, and borosilicate. Composite materials are composed of a matrix material reinforced with any of a variety of fibers made from ceramics, metals, or polymers. The reinforcing fibers are the primary load carriers of the material, with the matrix component transferring the load from fiber to fiber. Reinforcement of the matrix material may be achieved in a variety of ways. Fibers may be either continuous or discontinuous. Reinforcement may also be in the form of particles. Examples of composite materials include those made of carbon fibers, boron fibers, boron carbide fibers, carbon and graphite fibers, silicon carbide fibers, steel fibers, tungsten fibers, graphite/copper fibers, titanium and silicon carbide/titanium fibers. For purposes of the description of the invention where the inventive microporous thin film material is referred to as a microporous metal thin film, it will be understood to include both metal and pseudometallic materials.

The term “Elastic Deformation,” as used herein, is defined as a deformation caused by an applied load that is completely recoverable upon removal of the applied load. The elastic limit of a traditional metal is typically less than 1% strain.

The term “Plastic Deformation,” as used herein, is defined as deformation caused by an applied load that cannot be completely recovered upon removal of the load because bonds have been broken.

The term “Pseudoelastic Deformation,” as used herein, is defined as a deformation caused by an applied load that is completely recoverable upon removal of the load and the limit of which is characterized by being significantly larger than the elastic limit of a traditional metal (8% strain in the case of nitinol). This phenomenon is caused by a load or stress induced phase change that is reversible upon removal of the load.

The term “Pseudoplastic Deformation,” as used herein, is defined as a deformation caused by an applied load that requires some other action besides load removal, such as the application of heat, for complete recovery of the deformation. In pseudoplastic deformations, bonds have not been broken but, instead, have been reoriented (detwinned in the case of martensitic nitinol).

A stress-strain curve for austenitic nitinol in which a sample is taken all the way to failure at a temperature above A_{f} (finish of Austenitic transformation) can be separated into the following regions: elastic deformation of austenite, pseudoelastic deformation of austenite to stress induced martensite, elastic deformation of the stress induced martensite, plastic deformation of the stress induced martensite and fracture. Removal of the load at any point before the onset of plastic deformation of the stress induced martensite will result in complete recovery of the deformation.

Nitinol is in the thermally induced martensite state if the material deformed at temperatures below M_{f} (finish of Martensitic transformation) and subsequently kept below Aₛ (onset of austenitic transformation) or restrained from recovering its programmed shape above Aₛ. A stress-strain curve for martensitic nitinol in which a sample is taken all the way to failure at a temperature above below Aₛ can be separated into the following regions: elastic deformation of thermally induced martensite, pseudoplastic deformation of thermally induced martensite via detwinning, elastic deformation of the detwinned thermally induced martensite, plastic deformation of the detwinned thermally induced martensite and fracture. Removal of the load at any point before the onset of plastic deformation of the detwinned thermally induced martensite will result in complete recovery of the deformation when heated above A_{f}.

In a preferred embodiment of the present invention, the Aₛ temperature of the NiTi thin film microporous metal thin film covering is above body temperature. The microporous metal thin film covering is in a thermally induced martensite phase at its delivery diameter in a delivery catheter and, because the microporous metal thin film covering is approximately the same diameter as the ID of the catheter sheath, the microporous metal thin film covering experiences virtually no deformation while in the catheter. Upon delivery, the microporous metal thin film covering experiences a pseudoplastic radial deformation under the influence of shape memory expansion of the structural support.

In a preferred embodiment of the present invention, the A_{f} temperature of the NiTi structural support element is below body temperature. The structural support element is brought to a temperature below M_{f} and loaded into the catheter at a temperature below Aₛ such that the structural support element is in a thermally induced martensite phase before deformation from to the delivery diameter occurs. The structural support element is psueoplastically deformed during crimping and is considered to be in the pseudoplastically deformed, thermally induced martensite phase until deployment in the body by removing the constraining force at a temperature above A_{f}.

In accordance with each of the preferred embodiments of the present invention, shape memory metal or metal-like biocompatible materials which exhibit in vivo biological and mechanical responses substantially the same as biocompatible metals (hereinafter synonymously referred to as “pseudometals” or “pseudometallic materials”), are formed into functional devices by chemical vapor deposition of metal or metal-like chemical species onto a substrate capable of being subjected to chemical vapor deposition conditions, receiving a chemical vapor deposited film of a shape memory metal film or pseudometallic film thereupon and being released from the formed metal film.

The terms “metal film,” “thin metallic film” and “metal thin film” are used in this application synonymously to refer to single or plural layer films fabricated of biocompatible metals or biocompatible pseudometals having thicknesses greater than 0 μm and less than about 125 μm. When used as the structural support component, the thin metallic film preferably has a thickness greater than about 25 μm and when used as the covering component, the thin metallic film preferably has a thickness between 0.1 μm and 25 μm and most preferably between 0.1 μm and 10 μm.

In accordance with an alternate preferred embodiment of the present invention, shape memory polymers are deposited by low-temperature CVD. The shape memory effect exists for polymers (e.g. heat-shrinkable films). However, it is not a specific bulk property, but results from the polymer's structure and morphology. The effect is persistent in many polymers, which might differ significantly in their chemical composition. However only a few shape memory polymer systems have been described in the literature (Kim, et al., “Polyurethanes having shape memory effect,” Polymer 37(26):5781-93 (1996); Li et al., “Crystallinity and morphology of segmented polyurethanes with different soft-segment length,” J. Applied Polymer 62:631-38 (1996); Takahashi et al., “Structure and properties of shape-memory polyurethane block copolymers,” J. Applied Polymer Science 60:1061-69 (1996); Tobushi H., et al., “Thermomechanical properties of shape memory polymers of polyurethane series and their applications,” J. Physique IV (Colloque C1) 6:377-84 (1996)).

Examples of polymers used to prepare hard and soft segments of SMPs include various polyethers, polyacrylates, polyamides, polysiloxanes, polyurethanes, polyether amides, polyurethane/ureas, polyether esters, and urethane/butadiene copolymers. See, for example, U.S. Pat. No. 5,506,300 to Ward et al.; U.S. Pat. No. 5,145,935 to Hayashi; U.S. Pat. No. 5,665,822 to Bitler et al.; and Gorden, “Applications of Shape Memory Polyurethanes,” Proceedings of the First International Conference on Shape Memory and Superelastic Technologies, SMST International Committee, pp. 115-19 (1994). The SMPs that have been developed thus far appear to be limited to being able to hold only one temporary shape in memory. It would be advantageous to provide SMPs that are able to form objects which are able to hold more than one shape in memory.

The polymers incorporate “hard” and “soft” segments. The segments preferably are oligomers. As used herein, the term “oligomer” refers to a linear chain molecule having a molecular weight up to 15,000 Daltons. The polymers forming the segments are selected based on the desired glass transition temperature(s) (if at least one segment is amorphous) or the melting point(s) (if at least one segment is crystalline), which in turn is based on the desired applications, taking into consideration the environment of use. Preferably, the number average molecular weight of the polymer segment is greater than 400, and is preferably in the range of between 500 and 15,000.

The transition temperature at which the polymer abruptly becomes soft and deforms can be controlled by changing the monomer composition and the kind of monomer, which enables one to adjust the shape memory effect at a desired temperature. The thermal properties of the polymers can be detected, for example, by dynamic mechanical thermoanalysis or differential scanning calorimetry (DSC) studies. In addition the melting point can be determined using a standard melting point apparatus.

The polymers can be thermoset or thermoplastic, although thermoplastic polymers may be preferred due to their ease of molding. Thermosets, however, may be preferred in some applications, since they generally are softer than physically crosslinked polymer in their original shape at temperatures greater than Tₜᵣₐₙₛ. Preferably, the degree of crystallinity of the polymer or polymeric block(s) is between 3 and 80%, more preferably between 3 and 60%. When the degree of crystallinity is greater than 80% while all soft segments are amorphous, the resulting polymer composition has poor shape memory characteristics.

The tensile modulus of the polymers below the Tₜᵣₐₙₛ is typically between 50 MPa and 2 GPa (gigapascals), whereas the tensile modulus of the polymers above the Tₜᵣₐₙₛ is typically between 1 and 500 MPa. Preferably, the ratio of elastic modulus above and below the Tₜᵣₐₙₛ is 20 or more. The higher the ratio, the better the shape memory of the resulting polymer composition.

The polymer segments can be natural or synthetic, although synthetic polymers are preferred. The polymer segments can be biodegradable or non-biodegradable, although biodegradable polymer compositions generally are preferred for in vivo medical applications. In general, these materials degrade by hydrolysis, by exposure to water or enzymes under physiological conditions, by surface erosion, by bulk erosion, or a combination thereof. Non-biodegradable polymers used for medical applications preferably do not include aromatic groups, other than those present in naturally occurring amino acids.

The polymers are selected based on the desired glass transition temperature(s) (if at least one segment is amorphous) or the melting point(s) (if at least one segment is crystalline), which in turn is based on the desired applications, taking into consideration the environment of use. Preferably, the number average molecular weight of the polymer block is greater than 400, and is preferably in the range of between 500 and 15,000.

The polymer may be in the form of a hydrogel (typically absorbing up to about 90% by weight of water), and can optionally be ionically crosslinked with multivalent ions or polymers. Ionic crosslinking between soft segments can be used to hold a structure, which, when deformed, can be reformed by breaking the ionic crosslinks between the soft segments. The polymer may also be in the form of a gel in solvents other than water or aqueous solutions. In these polymers, the temporary shape can be fixed by hydrophilic interactions between soft segments.

Representative natural polymer blocks or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate)s, especially poly(β-hydroxybutyrate), poly(3-hydroxyoctanoate) and poly(3-hydroxyfatty acids). Representative natural biodegradable polymer blocks or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks or polymers include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymer segments include polyhydroxy acids, such as polylactides, polyglycolides and copolymers thereof; poly(ethylene terephthalate); polyanhydrides, poly(hydroxybutyric acid); poly(hydroxyvaleric acid); poly[lactide-co-(ε-caprolactone)]; poly[glycolide-co-(ε-caprolactone)]; polycarbonates, poly(pseudo amino acids); poly(amino acids); poly(hydroxyalkanoate)s; polyanhydrides; polyortho esters; and blends and copolymers thereof. Polymers containing labile bonds, such as polyanhydrides and polyesters, are well known for their hydrolytic reactivity. Their hydrolytic degradation rates can generally be altered by simple changes in the polymer backbone and their sequence structure.

Examples of non-biodegradable synthetic polymer segments include ethylene vinyl acetate, poly(meth)acrylic acid, polyamides, polyethylene, polypropylene, polystyrene, polyvinyl chloride, polyvinylphenol, and copolymers and mixtures thereof.

The polymers, monomers or polymer segments may be obtained from commercial sources such as Sigma Chemical Co., St. Louis, Mo.; Polysciences, Warrenton, Pa.; Aldrich Chemical Co., Milwaukee, Wis.; Fluka, Ronkonkoma, N.Y.; and BioRad, Richmond, Calif. Alternately, the polymers can be synthesized from monomers obtained from commercial sources, using standard techniques.

During deposition, the chamber pressure and temperature, the deposition pressure and the composition and partial pressure of the process gases are controlled to optimize deposition of the desired species onto the substrate. As is known in the microelectronic fabrication, nano-fabrication and vacuum coating arts, both the reactive and non-reactive gases are controlled and the inert or non-reactive gaseous species introduced into the deposition chamber is typically argon. The substrate may be either stationary or moveable; either rotated about its longitudinal axis, moved in an X-Y plane, planatarily or rotationally moved within the deposition chamber to facilitate deposition or patterning of the deposited material onto the substrate. The deposited material maybe deposited either as a uniform solid film onto the substrate, or patterned by (a) imparting either a positive or negative pattern onto the substrate, such as by etching or photolithography techniques applied to the substrate surface to create a positive or negative image of the desired pattern or (b) using a mask or set of masks which are either stationary or moveable relative to the substrate to define the pattern applied to the substrate. Patterning may be employed to achieve complex finished geometries of the resultant structural supports or microporous metal thin film covering, both in the context of spatial orientation of patterns of regions of relative thickness and thinness, such as by varying the thickness of the film over its length to impart different mechanical characteristics under different delivery, deployment or in vivo environmental conditions.

The device may be removed from the substrate after device formation by any of a variety of methods. For example, the substrate may be removed by chemical means, such as etching or dissolution, by ablation, by machining or by ultrasonic energy. Alternatively, a sacrificial layer of a material, such as carbon, aluminum or organic based materials, such as photoresists, may be deposited as an intermediate layer between the substrate and the structural support member and the sacrificial layer removed by melting, chemical means, ablation, machining or other suitable means to free the structural support member from the substrate.

The resulting device may then be subjected to post-deposition processing to modify the crystalline structure, such as by annealing, or to modify the surface topography, such as by etching to expose a heterogeneous surface of the device.

Alternate deposition processes which may be employed to form the structural support member in accordance with the present invention are cathodic arc, laser ablation, and direct ion beam deposition. As known in the metal fabrication arts, the crystalline structure of the deposited film affects the mechanical properties of the deposited film. These mechanical properties of the deposited film may be modified by post-process treatment, such as by, for example, annealing.

Materials most preferred for making the inventive biomaterials of the present invention by CVD processes are chosen for their biocompatibility, mechanical properties, i.e., tensile strength, yield strength, and their ease of deposition include, without limitation, the following: titanium, vanadium, aluminum, nickel, tantalum, zirconium, chromium, silver, gold, silicon, magnesium, niobium, scandium, platinum, cobalt, palladium, manganese, molybdenum and alloys thereof, such as zirconium-titanium-tantalum alloys, cobalt-chromium-molybdenum alloys, nitinol, and stainless steel.

In accordance with the method and material of the present invention vacuum deposited nitinol films having grain sizes within the range of 0.1-1 μm may be produced and exhibit optimal mechanical properties for the fabrication of stents, stent covers, grafts, and/or filter membranes. Thin walled nitinol tubes, such as tubes with diameters in the 1-16 mm range may be manufactured using the inventive technology with wall thicknesses between about 0.5-25 microns with wall thickness uniformity of about <10%. Sheets may be formed from tubes by cutting along the longitudinal axis of the tube, however, such sheets are readily fabricated in planar vacuum deposition systems. Prototype angioplasty balloons capable of being repeatedly inflated at pressures of several atmosphere's pressure may also be fabricated using the inventive methodology. The method of the present invention avoids using lubricants necessary in fabrication using cold working processes that contaminate heavily cold worked materials such as small diameter tubes like those used for cutting coronary stents. Finally, nitinol tubes having about 5% Ta added may be produced using the inventive method.

To deposit a NiTi film using a CVD method, it is preferable to first achieve an equilibrium temperature, then initiate a flow of an inert gas, such as argon (Ar), then initiate the flow of the reactant gases into the reactor. Two source gases are provided, a first source gas is titanium tetrachloride (TiCl₄) and a second source gas is nickel carbonyl, which will be decomposed or reduced to produce the Ni and Ti adsorbed species that will eventually produce the NiTi film.

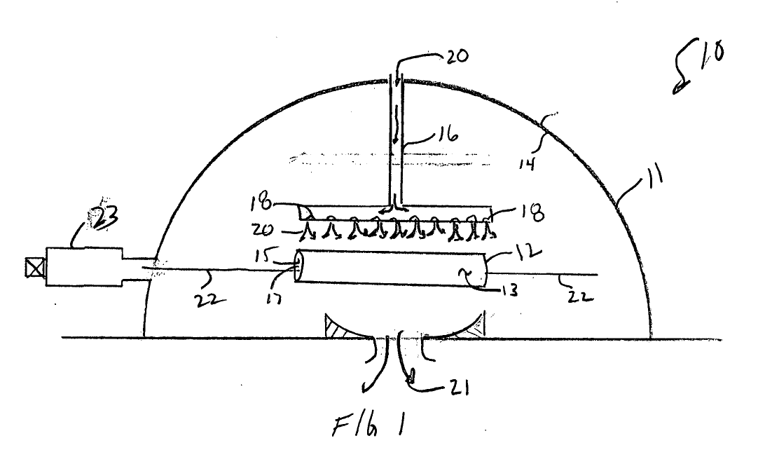

A prototypical CVD reactor 10 for fabricating the inventive devices is depicted in FIG. 1. The reactor 10 is illustrated without the requisite gas handling equipment, vacuum pumps, controls, interlocks, or other control equipment, such being well known to one of ordinary skill in the chemical vapor deposition art. Reactor 10 consists generally of a chamber 11 that can be a bell jar type, or tubular shape, and into which the workpiece or substrate 12 is introduced and supported by supports 22 in the chamber 11. The substrate 12 is introduced and withdrawn from the chamber 11 through a load lock 23 which permits maintenance of a vacuum within chamber 11 during substrate 12 handling. In accordance with the preferred embodiments of the present invention, substrate 12 is preferably either a generally tubular member having an outer wall surface 13 and an inner wall surface 15 and a central lumen 17 passing through the generally tubular member or a solid, generally cylindrical member. Chamber wall 14 defines the boundaries of the chamber, while a gas inlet conduit 16 is provided, which preferably has a plurality of gas inlet openings 18 to permit a flow of reactant gases 20 through the gas inlet conduit 16 and through the gas inlet openings 18 into the chamber 11 and permitted to deposit a reactant species onto the substrate 12. The non-reactant gas and by-products 20 are evacuated by a vacuum pump as is known in the art.

FIG. 2 depicts, generally, the CVD process 30 in accordance with the method of the present invention. A first step 32 entails identifying the appropriate reactant species for the desired film. Thus, for example if a nickel-titanium alloy film is desired, it will be necessary to identify appropriate source gases for nickel and for titanium, e.g., nickel carbonyl and titanium tetrachloride. Once the reactant gases have been identified and/or prepared, the CVD reactor is purged and pumped down to a desired vacuum at step 34, and the substrate introduced into the reaction chamber at step 36, preferably through a load lock chamber, as is known in the art. An inert gas, such as Argon, is flowed into the chamber first to allow the system to achieve its predetermined set points for pressure, substrate temperature and chamber temperature at step 38. The reactant gases are then introduced into the chamber at step 40, and permitted to decompose to produce the desired species at the substrate surface at step 42. After evacuating the non-reacting gases and byproducts and purging the reactor chamber, the substrate with the deposited film may be removed from the chamber at step 44. The deposited film is then preferably patterned on the substrate at step 46. The patterning step entails selective removal of portions of the deposited film to create either relatively thinner and thicker regions in the film, or void spaces or fenestrations in the deposited film which may be dimensioned and patterned to impart geometric deformability, provide openings to permit elution of a drug therethrough, or provide wells into which a drug may be placed for elution. Once patterned, the substrate is separated from the deposited film at step 48, preferably by chemically etching the substrate.

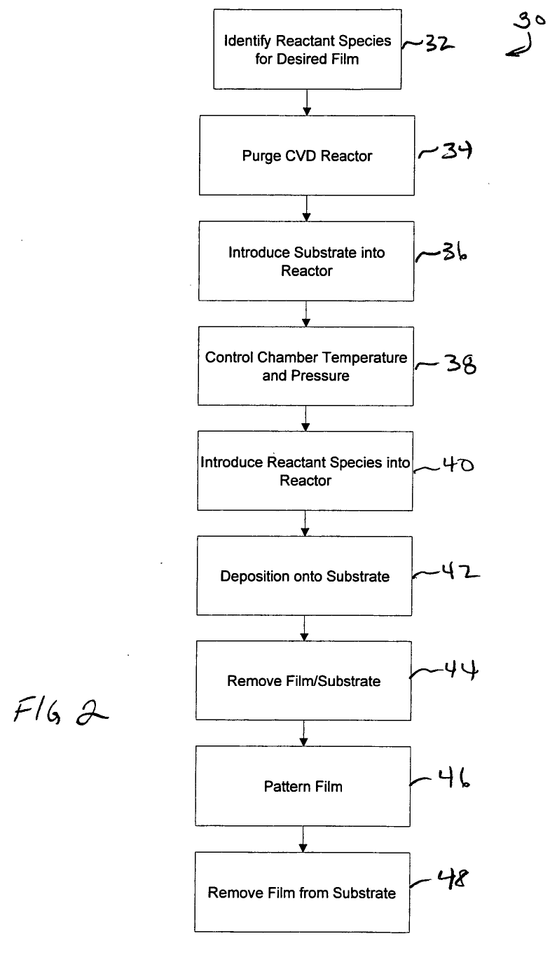

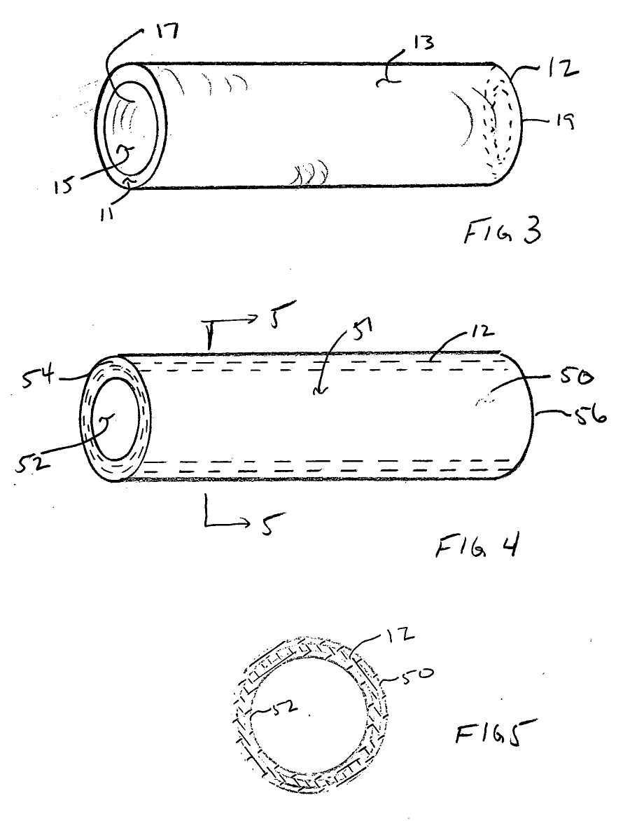

FIG. 3 depicts a generally tubular substrate 12 suitable for forming a tubular implantable medical device, such as a stent, stent cover, graft, filter membrane or other similar devices. The substrate 12 consists generally of a luminal wall surface 15, an abluminal wall surface 13, a central lumen 17 and opposing end surfaces 11 and 19. While the substrate 12 is depicted in an unpatterned state, it will be understood that patterned substrates 12, with recesses, depressions, projections or protuberances may be employed as are suitable in the chemical vapor deposition arts. In accordance with the preferred embodiment of the present invention, the substrate 12 is preferably fabricated of deoxygenated copper having a copper content greater than about 99.8%, stainless steel or other metals, silicon, silicon dioxide, silicon nitride or other suitable material capable of being removed from an encapsulating deposited film. Substrate selection must be compatible with the desired application of the deposited film. For removal by chemical etching, the principal constraint on substrate selection is that it has a high degree of selectivity for chemical etching over the material of the deposited film.

FIGS. 4 and 5 depict a CVD deposited film 50 on the generally tubular substrate 12. As will be understood by those of ordinary skill in the art, CVD deposits the reactant species onto all exposed surfaces of the substrate 12, and therefore conforms to the tubular shape of the substrate 12 yielding an abluminal deposited surface 51 on the abluminal surface 13 of the substrate 12, a luminal deposited surface 52 on the luminal surface 15 of the substrate 12, and deposited surfaces 54 and 56 at the opposing ends 15, 19 of the substrate 12.

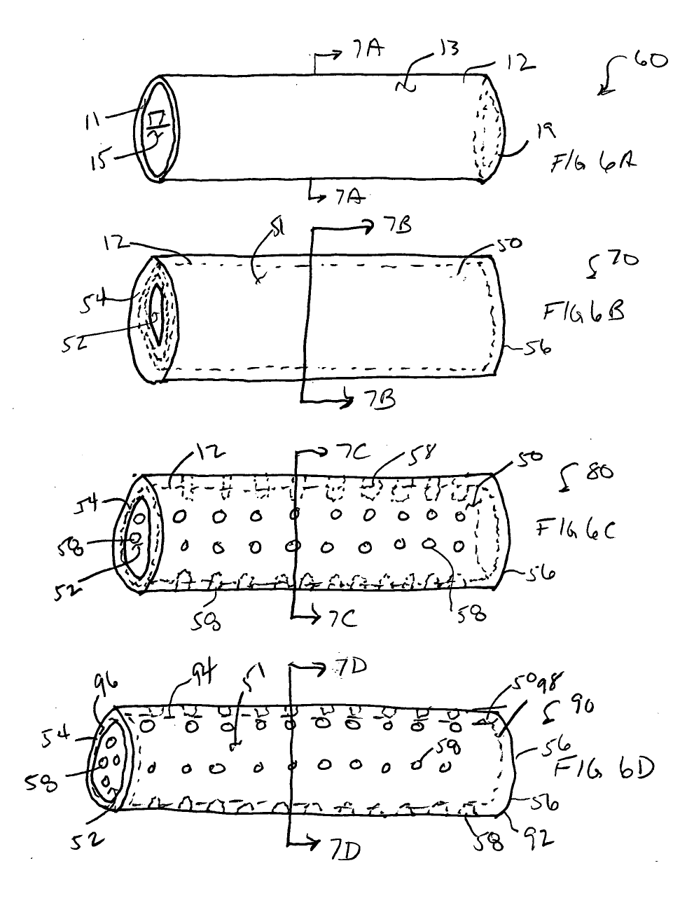

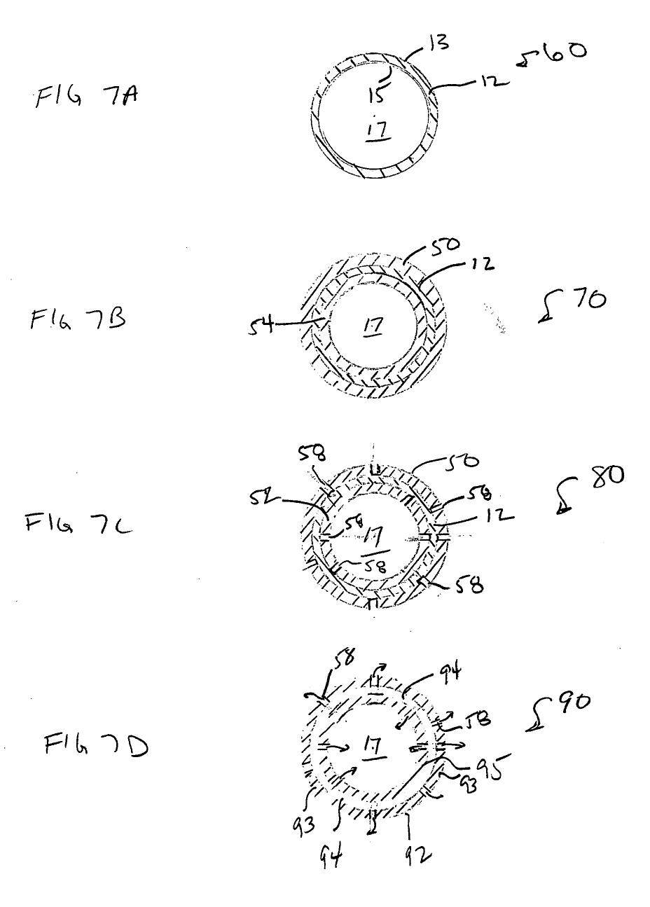

FIGS. 6A-6D, and their corresponding cross-sectional views 7A-7D, are sequential views depicting formation of a double-walled tubular drug-delivery device 90 in accordance with a second embodiment of the present invention. FIGS. 6A and 7A depict a tubular substrate 12 as described above with reference to FIG. 3. As shown in FIGS. 6B and 7B, after chemical vapor deposition, a deposited film 50 covers all exposed surfaces of the tubular substrate 12, as described above with reference to FIGS. 4 and 5.

To create an inventive double-walled drug delivery device 90 as illustrated in FIGS. 6C and 7C and 6D and 7D, after CVD, the substrate 12 and deposited film 50 are removed from the reactor, and a plurality of openings 58 are created through the deposited film 50, on the luminal surface 51, the abluminal surface 52, the first end surface 54 and/or the second end surface 56 of the deposited film. The openings 58 are preferably formed by laser ablating regions of the deposited film, but may also be formed by chemical or mechanical etching, or photolithographic techniques to selectively remove pre-defined areas of the deposited film to create wells, depots, or openings 58 which pass through the deposited film to the underlying substrate 12. The openings are preferably dimensioned to accommodate at least one of multiple purposes: 1) etching of the deposition substrate 12 through the openings 58, 2) drug elution, and 3) geometric deformation of the drug delivery device 90. The plurality of openings 58 is open at either of the luminal surface 51 or the abluminal surface 52, and each preferably has an open surface area between about 0.5 μm² to about 500 μm², with the total open surface area of all openings 58 at the luminal surface 51 and/or the abluminal surface 52 being between about 0.001 to 90%.

In accordance with the alternate preferred embodiment of the present invention there is provided a monolithic device 90 fabricated by CVD deposition of a device-forming material to form a coherent, monolithic structure capable of acting as a drug-delivery device in vivo. In accordance with the alternate preferred embodiment, and as illustrated in FIG. 6D, consists generally of a double walled tubular structure 92 having either a continuous or discontinuous plenum 94 intermediate luminal 95 and ablumenal walls 93 of the tubular structure 92. Significantly, the inventive tubular structure 92 also has proximal 54 and distal walls 56 which terminate opposing ends 96, 98 of the plenum 94. After the plurality of openings 58 are formed, the substrate 12 is removed by etching through the plurality of openings 58, to yield the plenum 94 intermediate luminal wall 95 and abluminal wall 93 of the double walled tubular structure 92. In the finished device, the plurality of openings 58 pass through either the luminal 95 and/or ablumenal walls 93 of the tubular structure 92 to allow for elution of a drug or drug combination from within plenum 94.

Local or localized delivery of drug or drug combinations may be utilized to treat a wide variety of conditions utilizing any number of medical devices, or to enhance the function and/or life of the device. Accordingly, in addition to the embodiments described herein, therapeutic or pharmaceutical agents may be added to any component of the device during fabrication to treat any number of conditions. In addition, therapeutic or pharmaceutical agents may be applied to the device, such as in the form of a drug or drug eluting layer, or surface treatment after the device has been formed. In a preferred embodiment, the therapeutic and pharmaceutical agents may include any one or more of the following: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) ll_{b}/lllₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligonucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

For such drugs the ideal pharmacokinetic profile will be one wherein the drug concentration reached therapeutic levels without exceeding the maximum tolerable dose and maintains these concentrations for extended periods of time till the desired therapeutic effect is reached. One of the ways such a profile can be achieved in an ideal case scenario would be by encapsulating the drug in a polymer matrix. The technology of polymeric drug delivery has been studied in details over the past 30 years and numerous excellent reviews are readily available to those skilled in the art. Polymeric drug delivery offers several advantages, including, for example: (1) Localized delivery of drug: The product can be implanted directly at the site where drug action is needed and hence systemic exposure of the drug can be reduced. This becomes especially important for toxic drugs which are related to various systemic side effects (such as the chemotherapeutic drugs). (2) Sustained delivery of drugs: The drug encapsulated is released over extended periods and hence eliminates the need for multiple injections. This feature can improve patient compliance especially for drugs for chronic indications, requiring frequent injections (such as for deficiency of certain proteins); (3) Stabilization of the drug: The polymer can protect the drug from the physiological environment and hence improve its stability in vivo. This particular feature makes this technology attractive for the delivery of labile drugs such as proteins.

The drug may be released from the polymer matrix either by diffusion out of the polymer matrix or by degradation of the polymer matrix of a combination of diffusion and degradation mechanisms. Polymer degradation may occur by enzymatic means, hydrolysis of a combination of these two. Hydrolysis, in turn, may be mediated by bulk erosion or by surface erosion of the polymer matrix. For a given drug, the release kinetics from the polymer matrix are predominantly governed by three factors, namely, the type of polymer, polymer morphology and the excipients present in the system.

The polymer could be non-degradable or degradable. A major disadvantage with non-degradable polymers is that a surgery may be required to harvest these polymers out of the body once they are depleted of the drug. Degradable polymers on the other hand do not require surgical intervention and hence are preferred for drug delivery applications. However, since they degrade to smaller absorbable molecules, it is important to make sure that the monomers are non-toxic in nature. Commonly employed polymers include, for example, polylactide (PLA), poly(lactide-co-glycolide) (PLGA), Poly(urethanes), Poly(siloxanes) or silicones, Poly(methyl methacrylate), Poly(vinyl alcohol), Poly(ethylene), Poly(vinyl pyrrolidone) and the specific polymers Poly(2-hydroxy ethyl methacrylate), Poly(N-vinyl pyrrolidone), Poly(methyl methacrylate), Poly(vinyl alcohol). Poly(acrylic acid). Polyacrylamide. Poly(ethylene-co-vinyl acetate). Poly(ethylene glycol). Poly(methacrylic acid).

Degradation of lactide based polymers and in general all hydrolytically degradable polymers, depends on the following properties: (1) chemical composition: The rate of degradation of polymers depends on the type of degradable bonds present on the polymer. In general, the rate of degradation of different chemical bonds follows as Anhydride>Esters>Amides; (2) crystallinity: generally, the higher the crystallinity of a polymer, the slower is its rate of degradation; and (3) hydrophilicity: if the polymer has a lot of hydrophobic groups present on it, then it is likely to degrade slower than a polymer which is hydrophilic in nature. Polylactides are known to be more hydrophobic as compared to PLGA and take a longer time to degrade. Among the polylactides, DL-PLA, which is a polymer of D and L-lactide, degrades faster than L-PLA, which is a homopolymer of L-lactide, presumably due to lesser crystallinity. Similarly, the more hydrophobic end-capped PLGA polymers degrade faster than the carboxyl-ended PLGA. Some new polymers showing promise as drug-delivery mechanisms include polyothroesters, polyphosphazenes, polyanhydrides and polyphosphoesters.

Morphology of the polymer matrix also plays an important role in governing the release characteristics of the encapsulated drug. The polymer matrix could be formulated as either micro/nano-spheres, gel, film or an extruded shape (such as cylinder, rod etc). The shape of the extruded polymer can be important to the drug release kinetics. For example, it has been shown that zero order drug release can be achieved using a hemispherical polymer form. Polymer microspheres are the most popular form due to manufacturing advantages as well as ease of administration (injectability by suspending in a vehicle). Polymer microspheres can be manufactured by using various techniques such as spray drying, solvent evaporation, etc. The type of technique used affects factors such as porosity, size distribution and surface morphology of the microspheres and may subsequently affect the performance of the drug delivery product.

Polymeric drug delivery products can be formulated with excipients added to the polymer matrix. The main objective of having excipients in the polymer matrix could be either to modulate the drug release, or to stabilize the drug or to modulate the polymer degradation kinetics. By incorporating basic salts as excipients in polymeric microspheres, the stability of the incorporated protein can be improved. It has been shown that these basic salts also slow the degradation of the polymer. Similarly, hydrophilic excipients can accelerate the release of drugs, though they may also increase the initial burst effect.

The present invention generally relates to a method of depositing shape memory or superelastic thin films by chemical vapor deposition (CVD) and devices made thereby. In particular, the invention relates to a method of depositing thin films whereby a thin film is deposited on a substrate surface using a CVD reaction in the production of a film of nickel-titanium shape memory or superelastic alloy. Such nickel-titanium-based shape memory or superelastic alloys may be binary nickel-titanium alloys or may include additional compounds to form ternary, quaternary, or higher level alloys.

The present invention may be employed to fabricate shape memory or superelastic devices by CVD, and, in particular, implantable medical devices including stents, stent-grafts, stent covers, grafts, occlusive and filter membranes and drug-delivery devices. Alternatively the present invention may also be employed to deposit superelastic, shape memory, elastically deformable materials or plastically deformable materials onto pre-existing devices, such as balloons, catheters, guidewires, stents, grafts or the like.

While the invention has been described with reference to its preferred embodiments, those of ordinary skill in the art will understand that variations in materials, process conditions, device configurations, film composition, and the like may be made without departing from the scope of the invention which is limited only by the claims appended hereto.

What is claimed is:

1. A method of fabricating shape memory or superelastic metal alloy films by chemical vapor deposition in a vacuum reactor, comprising the steps of:
a. Providing a substrate suitable for deposition of a shape memory or superelastic metal alloy thereupon;
b. Flowing a first gaseous species containing a first metal atom into the vacuum reactor;
c. Flowing a second gaseous species containing a second metal atom into the vacuum reactor;
d. Wherein the substrate temperature and vacuum pressure in the reactor are controlled in such a manner as to allow for dissociation of the first gaseous species and the second gaseous species from the gas phase to the solid phase and for co-deposition of a first metallic species from the first gaseous species and a second metallic species from the second gaseous species onto the heated substrate thereby forming a shape memory alloy film of the first metallic species and the second metallic species on the substrate.

2. The method according to claim 1, wherein the first gaseous species is selected from the group consisting of titanium halides.

3. The method according to claim 2, wherein the first gaseous species is titanium tetrachloride.

4. The method according to claim 1, wherein the second gaseous species is selected from the group of nickel carbonyl and bis-(cyclopentadienyl) nickel.

5. The method according to claim 1, wherein step (a) further comprises the step of providing a generally tubular substrate.

6. The method according to claim 5, wherein step (d) further comprises the step of forming the shape memory or superelastic alloy film on each of a luminal surface, and abluminal surface and opposing end surfaces of the generally tubular substrate.

7. The method according to claim 6, further comprising the step (e) of forming a plurality of openings passing through the shape memory alloy film to the generally tubular substrate.

8. The method according to claim 7, further comprising the step (f) of removing the generally tubular substrate through the plurality of openings, thereby forming a plenum within the shape memory or superelastic alloy film.

9. The method according to claim 8, further comprising the step (g) of loading at least one bioactive agent into the plenum through the plurality of openings.

10. The method according to claim 9, further comprising the step (h) of occluding the plurality of openings in such a manner as to provide for controlled elution of the at least one bioactive agent from the plenum and through the plurality of openings.

11. The method according to claim 1, wherein the substrate further comprises a catheter balloon.

12. The method according to claim 1, further comprising the step of patterning the shape memory or superelastic alloy film with a plurality of fenestrations.

13. The method according to claim 12, further comprising the step of annealing the patterned shape memory or superelastic alloy film after removal from the substrate.

14. A drug-eluting medical device, comprising a generally tubular member having an abluminal wall, a luminal wall, a first end and a second end, a plenum defined entirely between the abluminal wall, the luminal wall and the first and second ends, the abluminal wall, luminal wall, first end and second end being formed of a single, coherent, monolithic material and having a plurality of drug-elution openings passing through at least one of the abluminal wall, luminal wall, first end and second end in communication with the plenum.

15. The drug-eluting medical device according to claim 14, wherein the plurality of openings are present in at least one of the first end and second ends of the generally tubular member.

16. The drug-eluting medical device according to claim 14, further comprising a drug disposed within the plenum.

17. The drug-eluting medical device according to claim 16, further comprising an occlusive member associated with the plurality of openings that controls elution of the drug from the plenum through the plurality of openings.

18. The drug-eluting medical device according to claim 14, wherein the single, coherent, monolithic material is a shape memory material.

19. The drug-eluting medical device according to claim 14, wherein the single, coherent, monolithic material is a superelastic material.

20. The drug-eluting medical device according to claim 18, wherein the shape memory material is selected from the group consisting of metals, metal alloys and polymers.

21. The drug-eluting medical device according to claim 20, wherein the shape memory material is selected from the group consisting of titanium, vanadium, aluminum, nickel, tantalum, zirconium, chromium, silver, gold, silicon, magnesium, niobium, scandium, platinum, cobalt, palladium, manganese, molybdenum, stainless steel, nickel-titanium alloy, and chromium-cobalt alloy.
